(19) United States
(12) Patent Application Publication
(10) Pub. No.: US 2024/0233914 A1
(43) Pub. Date: Jul. 11, 2024

(54) PREDICTING MENTAL STATE CHARACTERISTICS OF USERS OF WEARABLE DEVICES

(71) Applicant: Hewlett-Packard Development Company, L.P., Spring, TX (US)

(72) Inventors: Jishang Wei, Guilford, CT (US); Rafael Antonio Ballagas, Palo Alto, CA (US); Erika H. Siegel, Palo Alto, CA (US)

(21) Appl. No.: 18/557,731

(22) PCT Filed: Apr. 29, 2021
(86) PCT No.: PCT/US2021/029853

§ 371 Date: Oct. 27, 2023

(51) Int. Cl. G16H 20/70; G02B 27/01; G06F 18/25; G16H 50/20

(52) U.S. Cl. CPC G16H 20/70; G02B 27/017; G06F 18/25; G16H 50/20

(57) ABSTRACT

An example method includes generating, with sensors of a wearable device, a plurality of physiological measures of a user of the wearable device. The method includes processing, with an inference engine of the wearable device, the plurality of physiological measures. The method includes generating a parametric distribution with the inference engine based on the processed physiological measures, wherein the parametric distribution includes a first parameter representing a predicted value of a current mental state characteristic of the user, and a second parameter representing an uncertainty quantification for the predicted value.

### BACKGROUND

Augmented reality (AR) systems and virtual reality (VR) systems may include a head-mounted display (HMD) that is tracked in a three-dimensional (3D) workspace. These systems allow the user to interact with a virtual world.

### BRIEF DESCRIPTION OF THE DRAWINGS

FIG. 1 is a block diagram illustrating elements of a wearable device according to an example.

FIG. 2 is a block diagram illustrating elements of an inference engine according to an example.

FIG. 3 is a diagram illustrating the sampling and labeling of physiological sensor data according to an example.

FIG. 4 is a diagram illustrating a graph of a cognitive load labels distribution for a set of training data according to an example.

FIG. 5 is a diagram illustrating a graph of inference engine cognitive load predictions for a testing dataset according to an example.

FIG. 6 is a flow diagram illustrating a method for predicting a current mental state characteristic of a user of a wearable device according to an example.

FIG. 7 is a block diagram illustrating a head mounted display according to an example.

FIG. 8 is a block diagram illustrating a non-transitory computer-readable storage medium according to an example.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Some examples disclosed herein are directed to a virtual reality headset with sensors to sense a plurality of physiological characteristics (e.g., pupillometry, eye movement, heart activities, etc.) of the user, and a cognitive load inference engine that generates a parametric distribution based on the sensed physiological characteristics. The parametric distribution may be a Gaussian distribution with parameters of mean and standard deviation. The mean value may represent a predicted value of a current mental state characteristic (e.g., cognitive load) of the user with the highest confidence, and the standard deviation may represent an uncertainty quantification for the predicted value, which indicates how uncertain the inference engine is about the prediction. In some examples, the bigger the standard deviation is, the more uncertain the inference engine may be about the prediction. In some examples, the inference engine provides calibration-free, real-time and continual point estimates of a cognitive load currently being experienced by a user, along with an uncertainty range for each of the cognitive load estimates. “Cognitive load” as used in some examples disclosed herein refers to the amount of mental effort for a person to perform a task or learn something new.

The training for the inference engine may involve collecting sensor readings from a training group of users while they perform tasks, and receiving their subjective ratings of experienced cognitive load. The collected data may be processed using a sliding window to generate a plurality of signal samples with associated labels. A set of features may be identified for each of the signal samples. The features may be processed using representation learning neural networks to generate learned representations of the data. The learned representations may be fused together into a fused representation, which is provided to another representation learning neural network for training.

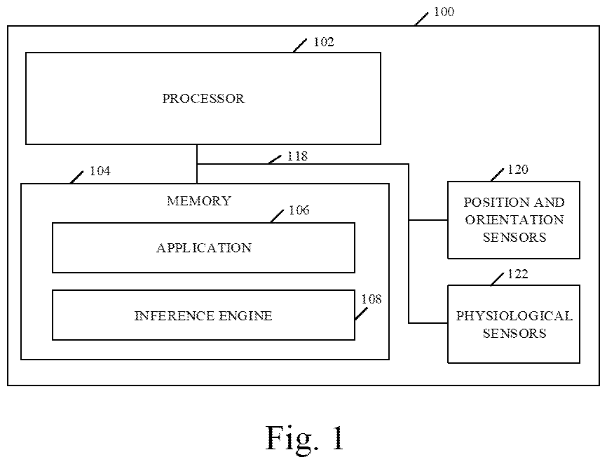

FIG. 1 is a block diagram illustrating elements of a wearable device 100 according to an example. In an example, wearable device 100 is a VR or AR headset or other head mounted display (HMD) device. Wearable device 100 includes at least one processor 102, memory 104, position and orientation sensors 120, and physiological sensors 122. In the illustrated example, processor 102, memory 104, and sensors 120 and 122 are communicatively coupled to each other via communication link 118.

Processor 102 includes a central processing unit (CPU) or another suitable processor. In one example, memory 104 stores machine readable instructions executed by processor 102 for operating the device 100. Memory 104 includes any suitable combination of volatile and/or non-volatile memory, such as combinations of Random Access Memory (RAM), Read-Only Memory (ROM), flash memory, and/or other suitable memory. These are examples of non-transitory computer readable storage media. The memory 104 is non-transitory in the sense that it does not encompass a transitory signal but instead is made up of at least one memory component to store machine executable instructions for performing techniques described herein.

Memory 104 stores application module 106 and inference engine module 108. Processor 102 executes instructions of modules 106 and 108 to perform some techniques described herein. Application module 106 generates a 3D visualization that is displayed by device 100. In an example, inference engine module 108 infers high-level insights about a user of device 100, such as cognitive load, emotion, stress, engagement, and health conditions, based on lower-level sensor data, such as that measured by physiological sensors 122. In an example, inference engine module 108 is based on a machine learning model that is trained with a training set of data to be able to predict a current cognitive load of a user along with an uncertainty quantification for that prediction. It is noted that some or all of the functionality of modules 106 and 108 may be implemented using cloud computing resources.

The device 100 may implement stereoscopic images called stereograms to represent a 3D visualization. The 3D visualization may include still images or video images. The device 100 may present the 3D visualization to a user via a number of ocular screens. In an example, the ocular screens are placed in an eyeglass or goggle system allowing a user to view both ocular screens simultaneously. This creates the illusion of a 3D visualization using two individual ocular screens. The position and orientation sensors 120 may be used to detect the position and orientation of the device 100 in 3D space as the device 100 is positioned on the user's head, and the sensors 120 may provide this data to processor 102 such that movement of the device 100 as it sits on the user's head is translated into a change in the point of view within the 3D visualization.

Although one example uses a VR headset to present the 3D visualization, other types of environments may also be used. In an example, an AR environment may be used where aspects of the real world are viewable in a visual representation while a 3D object is being drawn within the AR environment. Thus, much like the VR system described herein, an AR system may include a visual presentation provided to a user via a computer screen or a headset including a number of screens, among other types of devices to present the 3D visualization. Thus, the present description contemplates the use of not only a VR environment but an AR environment as well. Techniques described herein may also be applied to other environments.

In some examples, physiological sensors 122 are implemented as a multimodal sensor system that includes a plurality of different types of sensors to sense or measure different physiological or behavioral features of a user wearing the device 100. In some examples, physiological sensors 122 include a first sensor to track a user's pupillometry, a second sensor to track eye movement of the user, and a third sensor to track heart activities of the user (e.g., a pulse photoplethysmography (PPG) sensor). In other examples, physiological sensors 122 may include other types of sensors, such as an electromyography (EMG) sensor. Device 100 may also receive and process sensor signals from sensors that are not incorporated into the device 100.

In one example, the various subcomponents or elements of the device 100 may be embodied in a plurality of different systems, where different modules may be grouped or distributed across the plurality of different systems. To achieve its desired functionality, device 100 may include various hardware components. Among these hardware components may be a number of processing devices, a number of data storage devices, a number of peripheral device adapters, and a number of network adapters. These hardware components may be interconnected through the use of a number of busses and/or network connections. The processing devices may include a hardware architecture to retrieve executable code from the data storage devices and execute the executable code. The executable code may, when executed by the processing devices, cause the processing devices to implement at least some of the functionality disclosed herein.

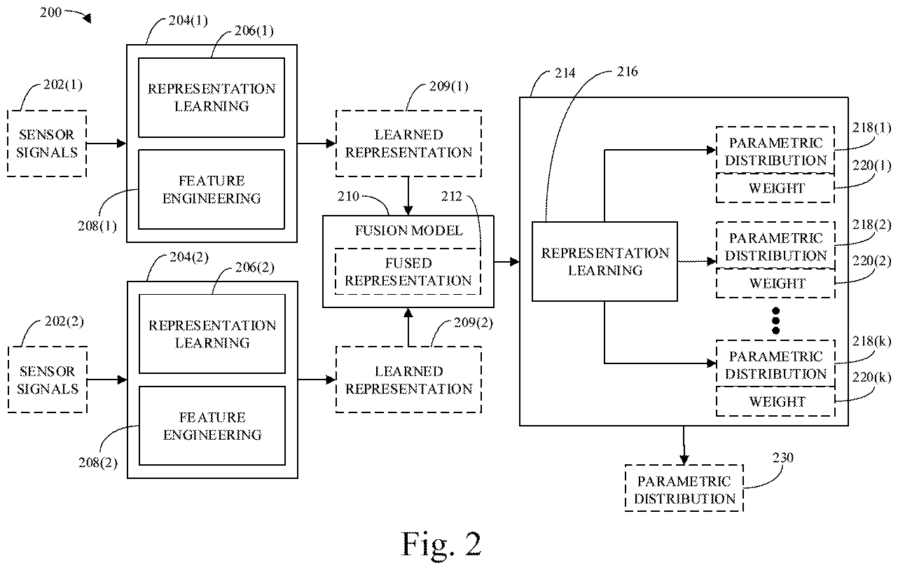

FIG. 2 is a block diagram illustrating elements of an inference engine 200 according to an example. In an example, inference engine module 108 (FIG. 1) is implemented with inference engine 200. Inference engine 200 includes a plurality of feature generation modules 204(1)-204(2) (collectively referred to as feature generation modules 204), a fusion model module 210, and a prediction module 214. The feature generation modules 204(1) and 204(2) include representation learning modules 206(1) and 206(2) (collectively referred to as representation learning modules 206), respectively, and feature engineering modules 208(1) and 208(2) (collectively referred to as feature engineering modules 208), respectively. Prediction module 214 includes representation learning module 216.

For the training of inference engine 200, a plurality of different tasks in a VR environment may be designed, which involve different levels of mental effort (e.g., low, medium, and high) to complete. In an example, the medium difficulty task may be a multitasking task that completely includes the low difficulty task, and the high difficulty task may be a multitasking task that completely includes the medium difficulty task. For example, the low difficulty task may be a visual vigilance task; the medium difficulty task may be the visual vigilance task and an arithmetic task; and the high difficulty task may be the visual vigilance task, the arithmetic task, and an audio vigilance task. Thus, in this example, higher level tasks are objectively harder than lower level tasks.

A training group of people may be recruited to perform the tasks. While each participant is performing the tasks, physiological sensor signals for the participant may be collected, such as the participant's pupillometry, eye movement, and heart activity information. These sensor signals are each a temporal series of data and are represented in FIG. 2 by sensor signals 202(1)-202(2) (collectively referred to as sensor signals 202). For each individual task performed by each participant, the participant may be asked after completion of the task to provide a subjective rating of the demanding cognitive load experienced by the participant during performance of the task. In an example, the subjective cognitive load experienced by the participant is a continuous value, c, falling in the range from 0 to 1, where 0 and 1 represent the lowest and highest experienced cognitive loads, respectively. In an example, for each task, each participant provides one subject cognitive load value for the entire task.

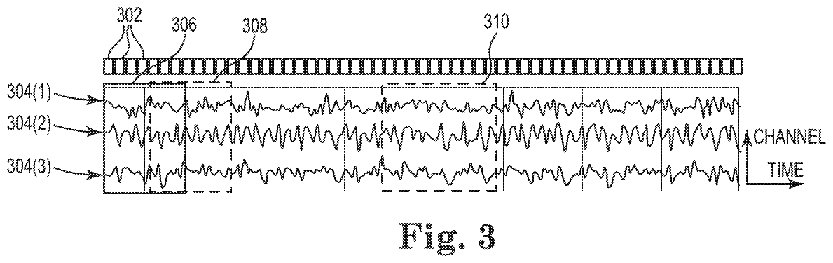

Thus, at this point in the training, physiological sensor signals 202 and labels (i.e., subjective ratings of cognitive load) will have been collected for each task performed by each of the participants. A next step in the training process is to process the physiological sensor signals 202 and labels. FIG. 3 is a diagram illustrating the sampling and labeling of physiological sensor data according to an example. FIG. 3 shows simplified representations of a plurality of different types of physiological sensor signals 304(1)-304(3) (collectively referred to as sensor signals 304) over time for a single task performed by a single participant. Sensor signals 304 are an example of sensor signals 202 (FIG. 2). A sliding window 306 may be used to generate signal samples from the sensor signals 304. In an example, the sliding window 306 has a width of 12.5 seconds and is moved across the sensor signals 304 with a one second skip step. Thus, as the sliding window 306 is moved across the sensor signals 304, it will reach position 308 and then position 310, and then eventually reach the end of the sensor signals 304. In an example, signal samples may be obtained individually from each of the sensor signals 304. A label is associated with each of the signal samples, as represented by labels 302 positioned above the sensor signals 304. Each label 302 represents the subjective cognitive load value experienced by the participant while completing the task, which, in an example, is a continuous value, c, falling in the range from 0 to 1.

In an example, each of the feature engineering modules 208 (FIG. 2) is associated with one of the sensor signals 202 and generates the signal samples and labels (e.g., labels 302 shown in FIG. 3) for its associated sensor signals 202. Each of the feature engineering modules 208 then generates a set of predefined features from each of the signal samples of the sensor signals 202 associated with that feature engineering module 208. In an example, each set of features is represented as an n-dimensional vector, v_n, where n represents the number of features. Each set of features may include various statistical, temporal, and frequency domain features, such as pupil diameters, blink, saccade, fixation, heart rate statistics, heart rate variabilities, respiration rate, and power spectral densities for PPG signals, as well as other features.

The n-dimensional vectors representing the sets of features associated with sensor signals 202(1) are provided to representation learning module 206(1) to generate a learned representation 209(1) corresponding to the sensor signals 202(1). The n-dimensional vectors representing the sets of features associated with sensor signals 202(2) are provided to representation learning module 206(2) to generate a learned representation 209(2) corresponding to the sensor signals 202(2). Learned representations 209(1) and 209(2) may be collectively referred to as learned representations 209. Each of the learned representations 209 represents a high-level representation of the sensor signal modality associated with that representation 209. The representation learning modules 206 may generate the learned representations 209 using representation learning neural networks, such as convolutional neural networks (CNNs) to extract local dependency patterns from input sequences. In an example, each of the learned representations 209 is an m-dimensional vector, v_m, where m represents the dimensionality of the signal representation. The representations 209 may be generated through a model that is trained separately through unsupervised learning.

Fusion model module 210 fuses the learned representations 209 into a fused representation 212, which is provided to representation learning module 216. In an example, fusion model module 210 uses a CNN to facilitate the determination of the fused representation 212. In an example, the representation learning module 216 includes a representation learning neural network that outputs parameters for a parametric distribution of possible prediction values based on the fused representation 212 provided as an input. In an example, the representation learning module 216 outputs k sets of parameters for a specific family of parametric distributions (e.g., k sets of means and standard deviations for Gaussian distributions), and k weight values. In an example, as shown in FIG. 2, representation learning module 216 outputs parameters for k parametric distributions 218(1)-218(k) (collectively referred to as parametric distributions 218) having associated weights 220(1)-220(k) (collectively referred to as weights 220), respectively. A weighted sum of the parametric distributions 218 may be generated for training using the weights 220.

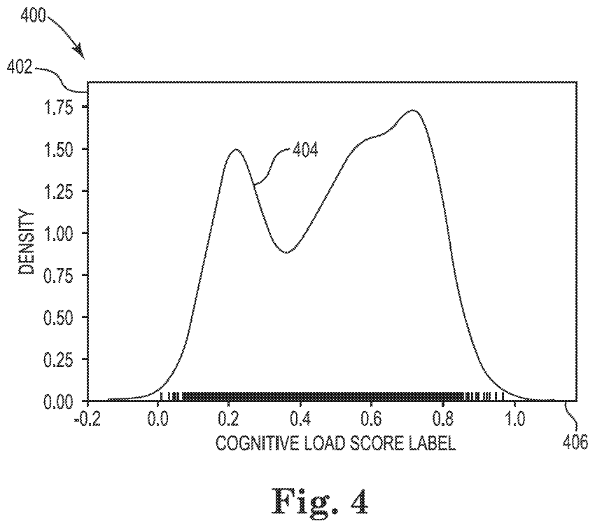

An object for the model training according to some examples is to maximize the likelihood of trained probabilistic models fitting in the distribution of target cognitive loads mapped from inputs. During training, the neural network weights from the representation learning modules 206 may be fixed, and the feature engineering modules 208 represent a set of deterministic algorithms/rules that have no weights to be tuned. In some examples, a couple of treatments may be applied for model training. One treatment is that the number, k, of parametric distributions 218 may be specified. The number, k, may be identified by a way of data exploration and by an understanding of the problem. For example, FIG. 4 is a diagram illustrating a graph 400 of a cognitive load labels distribution 404 for a set of training data according to an example. The horizontal axis 406 represents cognitive load score labels, and the vertical axis 402 represents density. In this case, it is known that the training involved three different task difficulty levels (i.e., low, medium, and high), and the distribution 404 includes three peaks. In this case, the number, k, of parametric distributions 218 may be specified as three. Another treatment is that, when calculating the loss function for the model to optimize, a “Winner Takes All” strategy may be used. This strategy, according to an example, means that the parametric distribution 218 that has the highest weight value 220 may be used to calculate the loss for a data input. The identified parametric distribution 218 that has the highest weight value 220 is represented in FIG. 2 by parametric distribution 230.

During inference according to an example, inputs of multiple modalities (e.g., sensor signals 202) may be sent to the inference engine 200, which will output a set of parametric probabilistic distributions 218 and their corresponding weights 220. The parametric distribution 218 with the highest weight 220 may be selected as the final prediction result, which is output by the prediction module 214 as parametric distribution 230. Using this distribution 230, the inference engine 200 can infer a single value cognitive load estimation. The variance of the distribution 230 may be used to quantify the prediction uncertainty. In examples in which distribution 230 is a Gaussian distribution, the mean value of the distribution 230 may be used as the cognitive load estimation result, and the standard deviation of the distribution may be used to measure the uncertainty of the prediction. The bigger the standard deviation is, the more uncertain the inference engine 200 may be about the prediction.

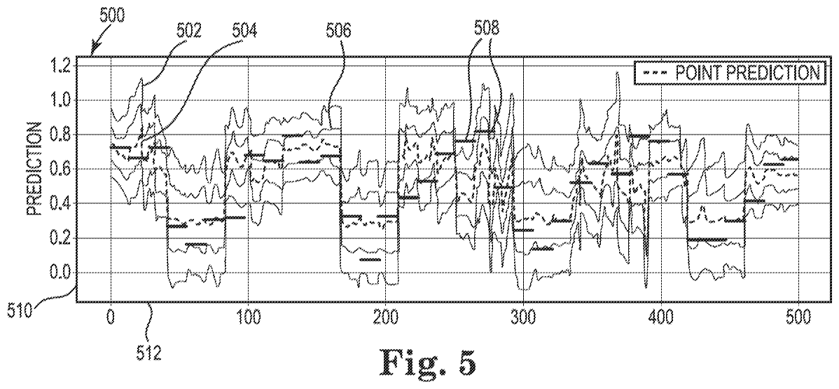

The result predicted by inference engine 200 may also be interpreted in another way. FIG. 5 is a diagram illustrating a graph 500 of inference engine cognitive load predictions for a testing dataset according to an example. The horizontal axis 512 represents time, and the vertical axis 510 represents prediction values. The horizontal lines segments 508 represent “ground truth” cognitive loads, and the curve 504 represents predicted cognitive loads using techniques described herein. The region 506 extends from above the curve 504 to below the curve 504 along the length of the curve 504 and represents a prediction interval that the final prediction will fall into with a 67% probability. In an example, the region 506 extends above the curve 504 by one standard deviation, and extends below the curve 504 by one standard deviation, so the region 506 represents a total of two standard deviations around the predicted result. The region 502 extends from above the region 506 to below the region 506 along the length of the region 506 and represents a prediction interval that the final prediction will fall into with a 95% probability. In an example, the region 502 extends above the curve 504 by two standard deviations, and extends below the curve 504 by two standard deviations, so the region 502 represents a total of four standard deviations around the predicted result. In some examples, inference engine 200 may output prediction intervals, such as those shown in FIG. 5.

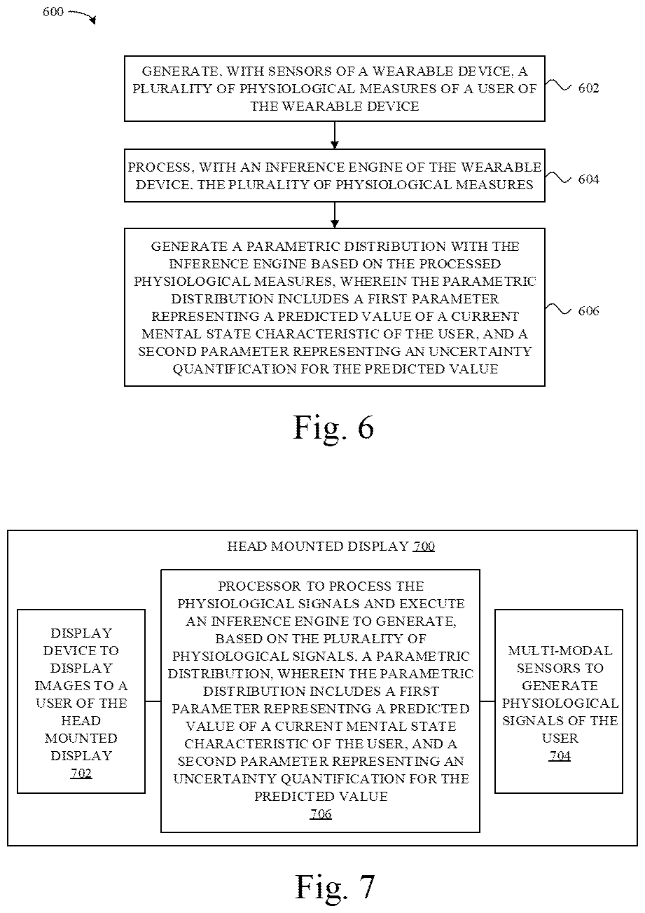

FIG. 6 is a flow diagram illustrating a method 600 for predicting a current mental state characteristic of a user of a wearable device according to an example. At 602, the method 600 includes generating, with sensors of a wearable device, a plurality of physiological measures of a user of the wearable device. At 604, the method 600 includes processing, with an inference engine of the wearable device, the plurality of physiological measures. At 606, the method 600 includes generating a parametric distribution with the inference engine based on the processed physiological measures, wherein the parametric distribution includes a first parameter representing a predicted value of a current mental state characteristic of the user, and a second parameter representing an uncertainty quantification for the predicted value.

In method 600, the current mental state characteristic may be a current cognitive load of the user. The parametric distribution may be a Gaussian distribution. The first parameter may be a mean value for the Gaussian distribution, and the second parameter may be a standard deviation for the Gaussian distribution. The wearable device may be a head mounted display, and the sensors may be multi-modal and may sense a plurality of different types of physiological measures of the user of the head mounted display. The physiological measures may include at least one of pupillometry information, eye movement information, and heart activity information. The processing may include: for each of the physiological measures, using a sliding window over time across the physiological measure to generate a plurality of signal segments corresponding to the physiological measure; for each of the physiological measures, extracting a set of features from each of the signal segments corresponding to the physiological measure; for each of the physiological measures, generating a learned representation corresponding to the physiological measure based on the set of features corresponding to the physiological measure; and fusing the learned representations for all of the physiological measures together to form a fused representation, and wherein the parametric distribution is generated with the inference engine based on the fused representation.

In method 600, the inference engine may be based on a trained machine learning model, and the method 600 may further include training the machine learning model, and the training may include: generating a plurality of physiological measures of each of a plurality of test set users of wearable devices while the test set users perform tasks of varying difficulty; receiving, from each of the test set users for each of the tasks, a subjective rating of the mental state characteristic experienced during that task; and performing a regression analysis based on the physiological measures and the subjective ratings to maximize a likelihood that a trained probabilistic model fits in a distribution of target mental state characteristic values. The regression analysis may include: generating a predetermined number of training probabilistic distributions for a given data input, wherein each of the training probabilistic distributions includes an associated weight; and calculating a loss function in a winner takes all manner using the training probabilistic distribution with a highest value for its associated weight.

FIG. 7 is a block diagram illustrating a head mounted display 700 according to an example. The head mounted display 700 includes a display device 702 to display images to a user of the head mounted display, and multi-modal sensors 704 to generate physiological signals of the user. The head mounted display 700 also includes a processor 706 to process the physiological signals and execute an inference engine to generate, based on the plurality of physiological signals, a parametric distribution, wherein the parametric distribution includes a first parameter representing a predicted value of a current mental state characteristic of the user, and a second parameter representing an uncertainty quantification for the predicted value.

The head mounted display 700 may be a virtual reality (VR) headset. In the head mounted display 700, the current mental state characteristic may be a current cognitive load of the user. In the head mounted display 700, the parametric distribution may be a Gaussian distribution, wherein the first parameter is a mean value for the Gaussian distribution, and wherein the second parameter is a standard deviation value for the Gaussian distribution.

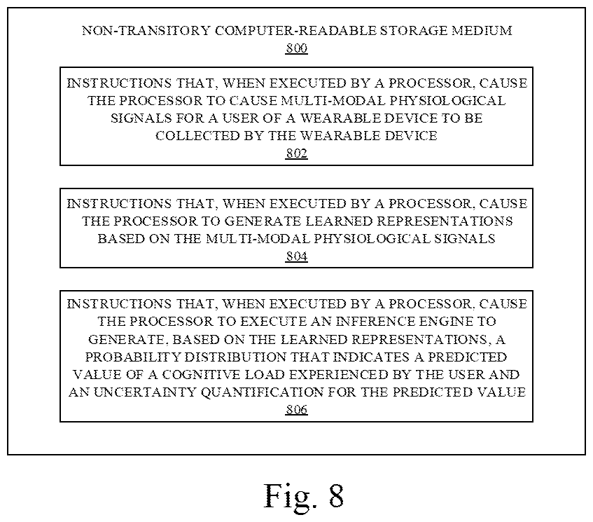

FIG. 8 is a block diagram illustrating a non-transitory computer-readable storage medium 800 according to an example. The non-transitory computer-readable storage medium 800 stores instructions 802 that, when executed by a processor, cause the processor to cause multi-modal physiological signals for a user of a wearable device to be collected by the wearable device. The non-transitory computer-readable storage medium 800 stores instructions 804 that, when executed by a processor, cause the processor to generate learned representations based on the multi-modal physiological signals. The non-transitory computer-readable storage medium 800 stores instructions 806 that, when executed by a processor, cause the processor to execute an inference engine to generate, based on the learned representations, a probability distribution that indicates a predicted value of a cognitive load experienced by the user and an uncertainty quantification for the predicted value.

For the non-transitory computer-readable storage medium 800, the probability distribution may be a Gaussian distribution, wherein a mean value for the Gaussian distribution indicates the predicted value the cognitive load, and wherein a standard deviation value for the Gaussian distribution indicates the uncertainty quantification for the predicted value.

Although some examples disclosed herein may involve inferences related to cognitive load, other examples may involve other types of inferences, such as stress, engagement, emotion, and others, including quantizing a prediction uncertainty for such inferences.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A method, comprising:
generating, with sensors of a wearable device, a plurality of physiological measures of a user of the wearable device;
processing, with an inference engine of the wearable device, the plurality of physiological measures; and
generating a parametric distribution with the inference engine based on the processed physiological measures, wherein the parametric distribution includes a first parameter representing a predicted value of a current mental state characteristic of the user, and a second parameter representing an uncertainty quantification for the predicted value.

2. The method of claim 1, wherein the current mental state characteristic is a current cognitive load of the user.

3. The method of claim 1, wherein the parametric distribution is a Gaussian distribution.

4. The method of claim 3, wherein the first parameter is a mean value for the Gaussian distribution, and wherein the second parameter is a standard deviation for the Gaussian distribution.

5. The method of claim 1, wherein the wearable device is a head mounted display, and wherein the sensors are multi-modal and sense a plurality of different types of physiological measures of the user of the head mounted display.

6. The method of claim 1, wherein the physiological measures comprise at least one of pupillometry information, eye movement information, and heart activity information.

7. The method of claim 1, wherein the processing comprises:
for each of the physiological measures, using a sliding window over time across the physiological measure to generate a plurality of signal segments corresponding to the physiological measure;
for each of the physiological measures, extracting a set of features from each of the signal segments corresponding to the physiological measure;
for each of the physiological measures, generating a learned representation corresponding to the physiological measure based on the set of features corresponding to the physiological measure; and
fusing the learned representations for all of the physiological measures together to form a fused representation, and wherein the parametric distribution is generated with the inference engine based on the fused representation.

8. The method of claim 1, wherein the inference engine is based on a trained machine learning model, wherein the method further comprises training the machine learning model, and wherein the training comprises:
generating a plurality of physiological measures of each of a plurality of test set users of wearable devices while the test set users perform tasks of varying difficulty;
receiving, from each of the test set users for each of the tasks, a subjective rating of the mental state characteristic experienced during that task; and
performing a regression analysis based on the physiological measures and the subjective ratings to maximize a likelihood that a trained probabilistic model fits in a distribution of target mental state characteristic values.

9. The method of claim 8, wherein the regression analysis comprises:
generating a predetermined number of training probabilistic distributions for a given data input, wherein each of the training probabilistic distributions includes an associated weight; and
calculating a loss function in a winner takes all manner using the training probabilistic distribution with a highest value for its associated weight.

10. A head mounted display, comprising:
a display device to display images to a user of the head mounted display;
multi-modal sensors to generate physiological signals of the user; and
a processor to process the physiological signals and execute an inference engine to generate, based on the plurality of physiological signals, a parametric distribution, wherein the parametric distribution includes a first parameter representing a predicted value of a current mental state characteristic of the user, and a second parameter representing an uncertainty quantification for the predicted value.

11. The head mounted display of claim 10, wherein the head mounted display is a virtual reality (VR) headset.

12. The head mounted display of claim 10, wherein the current mental state characteristic is a current cognitive load of the user.

13. The head mounted display of claim 10, wherein the parametric distribution is a Gaussian distribution, wherein the first parameter is a mean value for the Gaussian distribution, and wherein the second parameter is a standard deviation value for the Gaussian distribution.

14. A non-transitory computer-readable storage medium storing instructions that, when executed by a processor, cause the processor to:
cause multi-modal physiological signals for a user of a wearable device to be collected by the wearable device;
generate learned representations based on the multi-modal physiological signals; and
execute an inference engine to generate, based on the learned representations, a probability distribution that indicates a predicted value of a cognitive load experienced by the user and an uncertainty quantification for the predicted value.

15. The non-transitory computer-readable storage medium of claim 14, wherein the probability distribution is a Gaussian distribution, wherein a mean value for the Gaussian distribution indicates the predicted value the cognitive load, and wherein a standard deviation value for the Gaussian distribution indicates the uncertainty quantification for the predicted value.
